(19) United States
(12) Patent Application Publication
(10) Pub. No.: US 2005/0281761 A1
(43) Pub. Date: Dec. 22, 2005

(54) Methods of preventing UVB-induced skin damage

(76) Inventors: Michael Detmar, Boppelsen (CH); Satoshi Hirakawa, Ehime (JP); Seishiro Fujii, Boston, MA (US)

Correspondence Address: FISH & RICHARDSON PC, P.O. BOX 1022, MINNEAPOLIS, MN, 55440-1022, US

(21) Appl. No.: 11/096,451

(22) Filed: Apr. 1, 2005

Related U.S. Application Data
(60) Provisional application No. 60/559,300, filed on Apr. 1, 2004.

(51) Int. Cl. A61K 39/395; A61K 35/78; A61K 7/42

(52) U.S. Cl. 424/59; 424/145.1; 424/769; 424/766

(57) ABSTRACT

Skin damage, such as acute UVB-induced skin damage can be reduced in a subject, by administering to a subject having, or at risk for, acute UVB-induced skin damage, an agent that inhibits VEGF signaling.

### CROSS-REFERENCE TO RELATED APPLICATIONS

This application claims priority to U.S. Application Ser. No. 60/559,300, filed on Apr. 1, 2004, the entire contents of which are hereby incorporated by reference.

### BACKGROUND

Photodamage due to acute exposure to ultraviolet-B (UVB) irradiation can result, inter alia, in sunburn, inflammation, redness, swelling, blistering and edema of the skin.

### SUMMARY

Blocking vascular endothelial growth factor (VEGF) signaling can prevent acute UVB exposure-induced skin damage, e.g., UVB-induced sunburn, inflammation, redness, blistering, edema and/or swelling of the skin.

Accordingly, in one aspect, the invention features a method for identifying an agent that modulates, e.g., reduces, skin damage, e.g., radiation induced skin damage such as acute UVB-induced skin damage. The method includes identifying an agent that modulates, e.g., decreases, VEGF signaling (e.g., an agent that the reduces the expression, activity or levels of VEGF or of a VEGF receptor (VEGFR, preferably VEGFR-2)), and correlating the ability of an agent to modulate VEGF signaling, levels or activity with the ability to modulate skin damage, e.g., radiation induced skin damage such as acute UVB-induced skin damage. The method can further include selecting an identified agent, e.g., an agent that modulates skin damage.

In one embodiment, the agent is identified by evaluating the ability of a test agent to interact with, e.g., to bind, VEGF or VEGFR (e.g., VEGFR-2). In another embodiment, the agent is identified by evaluating the effect of a test agent to interact with a VEGF or VEGFR regulatory region, e.g., a promoter, e.g., a VEGF or a VEGFR promoter. In another embodiment, the agent is identified by evaluating the effect of the test agent on VEGF production in a skin cell, e.g., a keratinocyte. In another embodiment, the agent is identified by evaluating, e.g., quantitatively or qualitatively evaluating, the ability of a test agent to modulate acute VEGF signaling in a whole animal model, e.g., in a VEGF transgenic animal such as a VEGF overexpressing animal.

The test agent can be, e.g., a nucleic acid (e.g., an antisense, ribozyme), a polypeptide (e.g., an antibody or antigen-binding fragment thereof), a peptide fragment, a peptidomimetic, or a small molecule (e.g., a small organic molecule with a molecular weight of less than 2000 daltons). The test agent can be evaluated in a purified form, e.g., at least 10, 50, 70, 80, 90, or 99% pure, e.g., in a homogenous composition that does not include other test agents. In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics. The test agent can also be a crude or semi-purified extract, e.g., a botanical extract such as a plant extract, or algal extract.

The method can include correlating the effect of the agent on VEGF or VEGFR expression, levels, or activity, with a predicted effect of the agent on a mammal, e.g., a human, e.g., by providing (e.g., to the government, a health care provider, insurance company or patient) informational, marketing or instructional material, e.g., print material or computer readable material (e.g., a label, an email), related to the agent or its use, identifying the effect of the agent as a possible or predicted effect of the agent in a mammal, e.g., a human. E.g., the method can include identifying the agent as an agent that reduces acute UVB-induced skin damage, e.g., in humans, if it decreases VEGF or VEGFR expression, levels or activity, compared to a reference. The identification can be in the form of informational, marketing or instructional material, e.g., as described herein. In one embodiment, the method includes correlating a value for the effect of the agent with ability to reduce skin damage, e.g., generating a dataset correlating a value for the effect of the agent with ability to reduce skin damage.

In one embodiment, the method includes two evaluating steps, e.g., the method includes a first step of evaluating the test agent in a first system, e.g., a cell-free, cell-based, tissue system or animal model, and a second step of evaluating the test agent in a second system, e.g., a second cell or tissue system or in a non-human animal. In one embodiment, one of the evaluating steps includes evaluating the effect of the agent on a subject's skin or skin explant, e.g., evaluating the presence, extent or type of skin damage in the skin, preferably before and after acute UVB exposure. The subject can be an experimental animal or a human. In one embodiment, the first evaluation includes testing the effect of the test agent on a VEGF or VEGFR promoter that is linked to a heterologous sequence such as a reporter gene, and the second evaluation includes administering the test agent to a system, e.g., a cell based or animal system and evaluating effect of the agent on skin damage and/or VEGF production. In some embodiments, the method includes two evaluating steps in the same type of system, e.g., the agent is re-evaluated in a non-human animal after a first evaluation in the same or a different non-human animal. The two evaluations can be separated by any length of time, e.g., days, weeks, months or years.

In a preferred embodiment, the identifying step includes: (a) providing an agent to a cell, tissue or non-human animal whose genome includes an exogenous nucleic acid that includes a regulatory region of a VEGF or VEGFR gene (see, e.g., Gille et al., EMBO J. 1997 vol. 16(4):750-9 for VEGF promoter; and Giraudo et al., J Biol Chem. 1998 vol. 273(34):22128-35 for VEGFR-2 (flk1) promoter), operably linked to a heterologous sequence, e.g., a nucleotide sequence encoding a reporter polypeptide (e.g., a colorimetric (e.g., LacZ), luminometric, e.g., luciferase, or fluorescently detectable reporter polypeptide, e.g. GFP, EGFP, BFP, RFP); (b) evaluating the ability of a test agent to modulate the expression of the reporter polypeptide in the cell, tissue or non-human animal; and (c) selecting a test agent that modulates the expression of the reporter polypeptide as an agent that modulates acute UVB-induced skin damage.

In one embodiment, the animal is an experimental rodent. The animal can be wild type or a transgenic experimental animal, e.g., a VEGF transgenic rodent, e.g., a VEGF transgenic mouse described herein. The subject can also be a human. In a preferred embodiment, the evaluating step comprises administering the agent to the subject and evaluating skin damage (e.g., skin damage caused by acute exposure to UVB). In another embodiment, the cell or tissue is a skin cell, e.g., a keratinocyte; or tissue, e.g., a skin explant. In yet another embodiment, a cell, e.g., a skin cell, e.g., a keratinocyte, or a tissue, e.g., a skin explant, is derived from a transgenic animal.

In another aspect, the invention features a method of treating a subject, e.g., a human subject. The method includes (a) identifying a subject at risk for, or having, skin damage due to radiation exposure, e.g., acute UVB-exposure; and (b) administering to the subject an agent that modulates VEGF signaling in the subject, e.g., administering to the subject an effective amount of an agent that decreases the activity, level or expression of VEGF or VEGFR, e.g., an agent described herein. Preferably, the agent is administered to the subject's skin, e.g., topically. In a preferred embodiment, acute UVB-induced redness, inflammation, edema, blistering, swelling and/or sunburn of the skin are prevented or reduced. Acute UVB-exposure means exposure to at least one MED of UVB light, preferably at least 2, 3, or 5 MEDS. In one embodiment, the subject is exposed to between 3-8 MEDS, e.g., 3-5, 5-7, or 7-8 MEDS. In some embodiments, the subject will be, is, or has been, exposed to the sun when the UV index is moderate to extreme for a time sufficient to cause sunburn. The subject may exhibit one or more symptom of acute UVB exposure, e.g., skin inflammation, redness, swelling, blistering, tenderness or edema. In a preferred embodiment the subject is at least 5 years of age. Preferably, the subject is at least 10, 15, 20, 25, 30, 35, 40, 45, 50, or more years of age.

In a preferred embodiment, the agent is administered via a liposome carrier, e.g., a lecithin liposome or an alkylphospholipid liposome. The agent can be administered to the face, chest, neck, hands, and other regions of the body. The treatment can involve more than one administration, e.g., at least two, three, or four administrations, of the agent. The treatment can also involve daily administration of the agent.

In one embodiment, the method includes administering the agent in combination with a second treatment, e.g., a second treatment for skin, e.g., a sunscreen, antibiotic or moisturizer. In some embodiment, the agent is administered to the subject in combination with a controlled release device, e.g., a biocompatible polymer, micro particle, or mesh. The device can reduce degradation and control the release of the agent.

In some embodiments, the method includes evaluating the subject for skin damage. The evaluation can be performed before, during, and/or after the administration of the agent. For example, the evaluation can be performed at least 4 hours, 8 hours, 12 hours, 1 day, 2 days, 4, 7, 14, or more days before and/or after the administration.

In a preferred embodiment, the administration of an agent can be performed: prior to exposure to UVB light, e.g., prior to sun exposure; when acute-UVB induced skin damage (e.g., sunburn, selling, redness, and/or inflammation) is noticed or diagnosed; at the time a treatment for a skin damage-related disorder is begun or begins to exert its effects; or generally, as is needed to maintain skin health.

The period over which the agent is administered, or the period over which clinically effective levels are maintained in the subject, can be short term, e.g., for one day, two days, one week, or long term, e.g., for six months or more or a year or more, or short term, e.g., for less than a year, six months, one month, two weeks or less.

The identification of a subject in need of altered skin damage can be performed e.g., by the subject, by a health care provider, by a provider of a skin damage treatment, or another party. The agent may be administered, e.g., by the subject, by a health care provider, by a provider of a skin damage treatment, or another party. Likewise, the evaluation of the effect on skin damage may be performed, e.g., by the subject, by a health care provider, by a provider of a skin damage treatment, or another party.

An agent that decreases VEGF signaling to thereby decrease acute UVB-induced skin damage can be, for example: a VEGF binding protein or VEGFR-2 binding protein. For example, such binding proteins can bind and inhibit VEGF or bind and inhibit VEGFR-2 activity. The binding protein may inhibits the ability of VEGF or VEGFR-2 to interact with each other or another binding partner. In one embodiment, the binding protein is an antibody that specifically binds to VEGF or VEGFR-2, e.g., an antibody that disrupts VEGF's or VEGFR-2's ability to bind to a binding partner or to each other. Another exemplary agent is a mutated inactive VEGF that binds to VEGF or VEGFR-2 but disrupts VEGF signaling. Still another exemplary agent is VEGFR-2 (e.g., a non-signalling variant) or fragment thereof (e.g., an extracellular domain of VEGFR-2) that binds to VEGF or VEGFR-2 but disrupts VEGF signaling. Additional exemplary agents include a VEGF or VEGFR-2 nucleic acid molecule that can bind to a cellular VEGF or VEGFR-2 nucleic acid sequence, e.g., mRNA, and can inhibit expression of the protein, e.g., an antisense, siRNA molecule or ribozyme; an agent that decreases VEGF or VEGFR-2 gene expression, e.g., a small molecule that binds the promoter of VEGF or VEGFR-2 ; or a crude or semi-purified extract, e.g., a botanical extract such as a plant extract, or algal extract.

For example, subjects can be treated with VEGF antagonists, e.g., anti-VEGF antibodies such as bevacizumab; or VEGF receptor antagonists, e.g., anti-VEGF receptor antibodies or small molecule inhibitors, compounds having a molecular weight of less than 1500 daltons.

Exemplary inhibitors and VEGF receptor antagonists include inhibitors of VEGF receptor tyrosine kinase activity. 4-[4-(1-Amino-1-methylethyl)phenyl]-2-[4-(2-morpholin-4-yl-ethyl)phenylamino]pyrimidine-5-carbonitrile (JNJ-17029259) is one of a structural class of 5-cyanopyrimidines that are orally available, selective, nanomolar inhibitors of the vascular endothelial growth factor receptor-2 (VEGF-R2). Additional examples include: PTK-787/ZK222584(Astra-Zeneca), SU5416, SU11248 (Pfizer), and ZD6474 ([N-(4-bromo-2-fluorophenyl)-6-methoxy-7-[(1-methylpiperidin-4-yl)methoxy]quinazolin-4-amine]). Still other agents that can be used are broad specificity tyrosine kinase inhibitors, e.g., SU6668. See, e.g., Bergers, B. et al. (2003) J. Clin. Invest. 111, 1287-1295.

In another preferred embodiment, VEGF or VEGFR-2 is inhibited by decreasing the level of expression of an endogenous VEGF or VEGFR-2 gene, e.g., by decreasing transcription of the VEGF or VEGFR-2 gene. In a preferred embodiment, transcription of the VEGF or VEGFR-2 gene can be decreased by: altering the regulatory sequences of the endogenous VEGF or VEGFR-2 gene, e.g., by the addition of a negative regulatory sequence, such as a DNA-binding site for a transcriptional repressor, or by the removal of a positive regulatory sequence, such as an enhancer or a DNA-binding site for a transcriptional activator. In another preferred embodiment, the antibody that binds VEGF or VEGFR-2 is a monoclonal antibody, e.g., a humanized chimeric or human monoclonal antibody.

In a preferred embodiment, the agent that decreases VEGF expression is a botanical extract, e.g., pomegranate seed oil or grape seed oil.

The method can further include: increasing the activity of one or more anti-angiogenic factors, e.g., increasing the activity of naturally occurring anti-angiogenic proteins such as TSP-2 or TSP-1 in the subject. TSP-2 activity can be increased, e.g., by administering an agent which increases a TSP-2 activity. In a preferred embodiment, an agent which increases a TSP-2 activity can be one or more of the following: a TSP-2 polypeptide, or a biologically active fragment or analog thereof, e.g., a TSP-2 derived polypeptide or retro-inverso polypeptide thereof; a nucleic acid encoding a TSP-2 polypeptide, or a biologically active fragment or analog thereof; an agonist of TSP-2, e.g., an antibody or a small molecule having or increasing TSP-2 activity; or ban agent that increases TSP-2 nucleic acid expression, e.g., a small molecule which binds to the promoter region of TSP-2 and increases expression.

In a preferred embodiment, TSP-2 is increased by an agent, e.g., a small molecule, which induces TSP-2 expression. Examples of agents that can induce expression of TSP-2 include fetal calf serum and TGF-alpha. In preferred embodiments, an agent that induces TSP-2 expression is administered topically. In preferred embodiments, the agent is administered to a subject sufficiently before UVB exposure, e.g., sun exposure, such that an anti-angiogenesis effect is present in the subject's skin at the time of UVB exposure.

TSP-2 activity can also be increased by controlled delivery to the subject of a TSP-2 nucleic acid, or a TSP-2 protein, fragment, or analog. A TSP-2 nucleic acid, protein, fragment, or analog can be administered to the subject in combination with a controlled release device, e.g., a biocompatible polymer, micro particle, or mesh. The device can reduce degradation and control the release of the TSP-2 nucleic acid, protein, fragment, or analog. Such a TSP-2 biocompatible controlled release system can be administered to the subject, e.g., by injection or implantation, e.g., intramuscularly, subcutaneously, intravenously, or at an organ, joint cavity, or at a lesion.

The level of TSP-2 can also be increased by increasing the endogenous TSP-2 activity. Activity can be increased by increasing the level of expression of the gene, e.g., by increasing transcription of the TSP-2 gene; increasing the stability of the TSP-2 mRNA, e.g., by altering the secondary or tertiary structure of the mRNA; increasing the translation of TSP-2 mRNA, e.g., by altering the sequence of the TSP-2 mRNA; and/or increasing the stability of the TSP-2 protein. Transcription of the TSP-2 gene can be increased, e.g., by altering the regulatory sequences of the endogenous TSP-2 gene. In one embodiment the regulatory sequence can be altered by: the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the TSP-2 gene to be transcribed more efficiently.

In a preferred embodiment, the agent is a compound, e.g., small molecule, which induces TSP-2.

TSP-1 activity can be increased, e.g., by administering an agent which increases a TSP-1 activity. In a preferred embodiment, an agent which increases a TSP-1 activity can be one or more of the following: a TSP-1 polypeptide, or a biologically active fragment or analog thereof, e.g., a TSP-1 derived polypeptide or retro-inverso polypeptide thereof; a nucleic acid encoding a TSP-1 polypeptide, or a biologically active fragment or analog thereof; an agonist of TSP-1, e.g., an antibody or a small molecule having or increasing TSP-1 activity; or an agent that increases TSP-1 nucleic acid expression, e.g., a small molecule which binds to the promoter region of TSP-1 and increases expression.

In a preferred embodiment, TSP-1 is increased by an agent, e.g., a small molecule, which induces TSP-1 expression. Examples of agents that can induce expression of TSP-1 include fetal calf serum and TGF-alpha. In preferred embodiments, an agent that induces TSP-1 expression is administered topically. In preferred embodiments, the agent is administered to a subject sufficiently before UVB exposure, e.g., sun exposure, such that an anti-angiogenesis effect is present in the subject's skin at the time of UVB exposure.

TSP-1 activity can also be increased by controlled delivery to the subject of a TSP-1 nucleic acid, or a TSP-1 protein, fragment, or analog. A TSP-1 nucleic acid, protein, fragment, or analog can be administered to the subject in combination with a controlled release device, e.g., a biocompatible polymer, micro particle, or mesh. The device can reduce degradation and control the release of the TSP-1 nucleic acid, protein, fragment, or analog. Such a TSP-1 biocompatible controlled release system can be administered to the subject, e.g., by injection or implantation, e.g., intramuscularly, subcutaneously, intravenously, or at an organ, joint cavity, or at a lesion.

The method can further include: increasing activity of interferon beta, e.g., by administering an interferon beta, e.g., beta1 or beta2.

In another aspect, the invention features compositions containing an agent, e.g., an agent described herein, e.g., an agent identified by a screening method described herein, that decreases the expression, activity, or level of VEGF or VEGFR (e.g., VEGFR-2), for reducing acute UVB-induced skin damage. In a preferred embodiment, the composition is a cosmetic composition, e.g., formulated for topical administration. In a preferred embodiment, the composition also has a fragrance, a preservative, or other cosmetic ingredient, e.g., a moisturizer, or sunscreen agent, e.g., octyl methoxycinnamate, aminobenzoic acid, oxybenzone, padimate O, homosalate, or titanium dioxide. The composition can be provided in a shampoo, oil, cream, lotion, soap, foam, gel, or other cosmetic preparation. In a preferred embodiment, the composition also has a cosmetic ingredient, e.g., a fragrance or moisturizer.

In another aspect, the invention features a method of modulating skin damage in a subject. The method includes supplying to the subject a composition containing an agent that affects the expression, activity or level of a component of VEGF or VEGFR (e.g., VEGFR-2), e.g., an agent described herein, e.g., an agent identified by a screening method described herein, and supplying to the subject instructions for application of the agent, e.g., to treat skin damage such as acute UVB-induced skin damage.

In another aspect, the invention features a kit for modulating skin damage of a subject that includes a composition described herein, e.g., a composition containing an agent that affects the expression, activity, or level of a component of VEGF or VEGFR (e.g., VEGFR-2); and instructions for use, e.g., instructions to apply the composition to an area of the body in need of treatment for acute UVB-induced skin damage, e.g., redness, swelling, sunburn and/or inflammation. In a preferred embodiment, the composition also has a cosmetic ingredient, e.g., a fragrance or moisturizer.

An effective amount of the agent of the present invention is defined as the amount of a composition that, upon administration to a subject (e.g., a human), reduces skin damage in the subject. The effective amount to be administered to a subject is typically based on a variety of factors including age, sex, surface area, weight, and conditions of the skin. Body surface area may be approximately determined from height and weight of the patient. See, e.g., Scientific Tables, Geigy Pharmaceuticals, Ardley, N.Y., 1970, 537. Effective doses will vary, as recognized by those skilled in the art, dependent on route of administration, excipient usage, and the possibility of co-usage with other treatments such as usage of other skin damage-modulating compounds.

In another aspect, the invention features a method that includes: applying, to a skin region of a human subject, (e.g., forearms, legs, back, torso, head, face, scalp, a protective amount of a VEGF signalling inhibitor; and exposing the subject to irradiation, e.g., to sunlight, e.g., direct or high intensity sunlight, or to a UV-light, e.g., as in a tanning parlor. A protective amount is an amount sufficient to reduced skin damage to a detectable or statistically significant level In another aspect, the invention features a method of monitoring a subject or cells from a subject, e.g., skin cells. The method include evaluating expression of one or more of VEGF, TSP-1 and interferon beta. An increase in VEGF levels and a decrease in TSP-1 or interferon beta, relative to a reference (e.g., control or un-irradiated cells) can indicate that the subject or cells from the subject are at risk for or have been exposed to a skin damaging condition, e.g., irradiation, e.g., UVB irradiation.

The details of one or more embodiments of the invention are set forth in the accompanying drawings and the description below. Other features, objects, and advantages of the invention will be apparent from the description and drawings, and from the claims.

As used herein, exposure to acute UVB-radiation means exposure to natural sunlight or artificial UVB radiation (e.g., a UVB sun lamp, e.g., for tanning, or for phototherapy, e.g., for treatment of psoriasis, atopic dermatitis, or vitiligo) of at least one MED.

As used herein “VEGF” refers to VEGF-A (including isoforms thereof) or Vascular permeability factor (VPF). See, e.g., Gille et al., supra. However, in some implementations other related proteins, VEGF-B and VEGF-C can be used.

### DETAILED DESCRIPTION

Acute UVB irradiation of the skin results in erythema, vasodilation, enhanced vascular permability and edema formation. To investigate the biological importance of VEGF for the acute cutaneous UVB response, VEGF overexpressing transgenic mice and their wild-type litter mates were exposed to acute and chronic UVB irradiation. VEGF overexpressing mice were characterized by a two-fold lower minimal erythema dose (MED=2 min), as compared with wild-type mice (MED=4 min), after a single UVB exposure. Acute UVB irradiation for 2 min induced proliferation of epidermal keratinocytes and endothelial cells, dermal edema, vascular dilation, and inflammatory cell infiltration in the skin of VEGF transgenic mice but not in wild-type mice. Thus, among other things, VEGF may mediate cutaneous damage that occurs after UVB irradiation.

Systemic treatment with an anti-VEGF blocking antibody reduced the sensitivity of wild-type mice to acute UVB irradiation. Thus, the VEGF/VEGF receptor pathway is a novel target for the prevention of acute photodamage of the skin.

Acute UVB Damage

The UV index (developed by the Environmental Protection Agency) indicates the intensity of the sun's UV rays on a given day. There are four categories—moderate (UV index is less than 3), high (UV index is 3 to 6) very high (UV index is 6 to 10) and extreme (UV index is greater than 10). A moderate UV Index means it will take more than an hour to burn your skin; an extreme level means it will take less than 15 minutes. The index is often included with weather reports. Clinically, UVB exposure is measured in minimal erythema doses (MED's). One MED is the amount of UVB required to produce a sunburn in sensitive skin. Moderate-to-severe acute UVB-induced skin damage, e.g., sunburn, can occur at 3-8 MEDs.

Screening Methods

Numerous methods exist for evaluating whether an agent can modulate VEGF signaling, e.g., VEGF or VEGFR gene expression, activity or level. In one embodiment, the ability of a test agent to modulate, e.g., increase or decrease, e.g., permanently or temporarily, expression from a VEGF or VEGFR (e.g., VEGFR-2) gene promoter is evaluated by e.g., routine reporter (e.g., LacZ or GFP or luciferase) transcription assay. For example, a cell or transgenic animal whose genome comprises a reporter gene operably linked to a VEGF or VEGFR (e.g., VEGFR-2) promoter, can be contacted with a test agent, and the ability of the test agent to increase or decrease reporter activity is indicative of the ability of the agent to modulate acute UVB skin damage. In another embodiment, the ability of a test agent to modulate VEGF or VEGFR (e.g., VEGFR-2) gene expression, or VEGF or VEGFR (e.g., VEGFR-2) activity or level, is evaluated in a transgenic animal, for example, the transgenic animal described herein.

The effect of a test agent on VEGF or VEGFR (e.g., VEGFR-2) gene expression or VEGF or VEGFR (e.g., VEGFR-2) activity or level may also be evaluated in a cell, cell lysate, or subject, preferably a non-human experimental mammal, and more preferably a rodent (e.g., a rat, mouse, or rabbit), or explant (e.g., skin) thereof. Methods of assessing VEGF or VEGFR (e.g., VEGFR-2) gene expression are well know in the art, e.g., Northern analysis, ribonuclease protection assay, reverse transcription-polymerase chain reaction (RT-PCR) or RNA in situ hybridization (see, e.g., Sambrook et al. Molecular Cloning: A Laboratory Manual (3^{rd} ed. 2001)). The level of VEGF or VEGFR (e.g., VEGFR-2) may be monitored by, e.g., Western analysis, immunoassay, or in situ hybridization. VEGF or VEGFR (e.g., VEGFR-2) activity, e.g., altered promoter binding and/or transcription activity, may be determined by, e.g., electrophoretic mobility shift assay, DNA footprinting or reporter gene assay. Preferably, the effect of a test agent on VEGF or VEGFR (e.g., VEGFR-2) gene expression or VEGF or VEGFR (e.g., VEGFR-2) activity or level is observed as a change in skin damage in a subject. More preferably, the effect of a test agent on VEGF or VEGFR (e.g., VEGFR-2) gene expression or VEGF or VEGFR (e.g., VEGFR-2) activity or level is evaluated on a transgenic cell or non-human animal, or explant or cell derived therefrom, having altered VEGF signaling, as compared to a wild-type cell or non-human animal, or explant or cell derived therefrom.

The test agent may be administered to a cell, cell extract, explant or subject expressing a transgene comprising the VEGF or VEGFR (e.g., VEGFR-2) gene promoter fused to LacZ. (Enhancement or inhibition of transgene, e.g., a reporter, e.g., LacZ or GFP, transcription, as a result of an effect of the test agent on the VEGF or VEGFR (e.g., VEGFR-2) gene promoter or factors regulating transcription from the VEGF or VEGFR (e.g., VEGFR-2) gene promoter, may be easily observed as a change in color. Reporter transcript levels, and thus VEGF or VEGFR (e.g., VEGFR-2) gene promoter activity, may be monitored by established methods, e.g., Northern analysis, ribonuclease protection assay, reverse transcription-polymerase chain reaction (RT-PCR) or RNA in situ hybridization (see, e.g., Cuncliffe et al. (2002) Mamm. Genome 13:245). Agents may be evaluated using a cell-free system, e.g., an environment comprising the VEGF or VEGFR (e.g., VEGFR-2) gene promoter-reporter transgene (e.g., VEGF or VEGFR (e.g., VEGFR-2) gene promoter-LacZ transgene), transcription factors binding the VEGF or VEGFR (e.g., VEGFR-2) gene promoter, a crude cell lysate or nuclear extract, and the test agent (e.g., an agent described herein), wherein an effect of the agent on VEGF or VEGFR (e.g., VEGFR-2) gene promoter activity is detected as a color change.

Exemplary Agents

A variety of agents can be used as a VEGF/VEGFR antagonists to treat or prevent skin damage. The agent may be any type of compound that can be administered to a subject (e.g., antibodies, proteins, peptides, glycoproteins, glycopeptides, glycolipids, polysaccharides, oligosaccharides, nucleic acids, bioorganic molecules, peptidomimetics, pharmacological agents and their metabolites, transcriptional and translation control sequences, and the like). In one embodiment, the VEGF/VEGFR antagonist is a biologic, e.g., a protein having a molecular weight of between 5-300 kDa.

For example, a VEGF/VEGFR antagonist may inhibit binding of VEGF to an VEGFR or may prevent VEGF-mediated signal transduction, e.g., as transduced by the VEGFR protein, e.g., VEGFR-2. A VEGF/VEGFR modulator that binds to VEGF may alter the conformation of VEGF, hinder binding of VEGF to VEGFR, or otherwise decrease the affinity of VEGF for a VEGFR or prevent the interaction between VEGF and a VEGFR.

A VEGF/VEGFR modulator (e.g., an antibody) may bind to VEGF or to a VEGFR with a Kd of less than 10⁻⁶, 10⁻⁷, 10⁻⁸, 10⁻⁹, or 10⁻¹⁰ M. In one embodiment, the VEGF/VEGFR modulator binds to VEGF (i.e., VEGF-A with an affinity at least 5, 10, 20, 50, 100, 200, 500, or 1000 better than its affinity for a non-VEGF-A protein, e.g., VEGF-B or VEGF-C. A preferred VEGF/VEGFR modulator specifically binds VEGF or VEGFR, such as a VEGF or VEGFR specific antibody.

Exemplary VEGF/VEGFR modulators include antibodies that bind to VEGF or VEGFR and soluble forms of the VEGFR that compete with cell surface VEGFR for binding to VEGF. An example of a soluble form of the VEGFR is a protein that includes at least a portion of the extracellular domain of VEGFR (e.g., a soluble VEGF-binding fragment of VEGFR). The protein can further include other sequences, e.g., heterologous sequences such as an Fc domain. Other soluble forms of VEGFR, e.g., forms that do not include an Fc domain, can also be used.

An exemplary soluble form of the VEGFR protein includes a region of the VEGFR protein that binds to VEGF, e.g., an extracellular domain, e.g., domain of in the extracellular region. This region can be physically associated, e.g., fused to another amino acid sequence, e.g., an Fc domain, at its N— or C-terminus. The region from VEGFR can be spaced by a linker from the heterologous amino acid sequence.

Exemplary VEGF/VEGFR modulators include antibodies that bind to VEGF and/or VEGFR. In one embodiment, the antibody inhibits the interaction between VEGF and a VEGFR, e.g., by physically blocking the interaction, decreasing the affinity of VEGF and/or VEGFR for its counterpart, disrupting or destabilizing VEGF complexes, sequestering VEGF or a VEGFR, or targeting VEGF or VEGFR for degradation. In one embodiment, the antibody can bind to VEGF or VEGFR at an epitope that includes one or more amino acid residues that participate in the VEGF/VEGFR binding interface. Such amino acid residues can be identified, e.g., by alanine scanning. In another embodiment, the antibody can bind to residues that do not participate in the VEGF/VEGFR binding. For example, the antibody can alter a conformation of VEGF or VEGFR and thereby reduce binding affinity, or the antibody may sterically hinder VEGF/VEGFR binding.

In addition to antibodies that bind to VEGF and/or VEGFR, other antibodies can be used. In one embodiment, the antibody can prevent activation of a VEGF/VEGFR mediated event or activity.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A., et al. (1991) Sequences of Proteins of Immunological Interest, Fifth Edition, US Department of Health and Human Services, NIH Publication No. 91-3242; and Chothia, C. et al. (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N— or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with VEGFR.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

Antibodies that bind to VEGF or a VEGFR can be generated by a variety of means, including immunization, e.g., using an animal, or in vitro methods such as phage display. All or part of VEGF or VEGFR can be used as an immunogen or as a target for selection. For example, VEGF or a fragment thereof, VEGFR or a fragment thereof, can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Accordingly, by using hybridoma technology, at least partly human, antigen-specific monoclonal antibodies with the desired specificity can be produced and selected. See, e.g., XENOMOUSE™, Green et al. (1994) Nat. Gen. 7:13-21; US 2003-0070185; U.S. Pat. No. 5,789,650; and WO 96/34096.

Non-human antibodies to VEGF and VEGFR can also be produced, e.g., in a rodent. The non-human antibody can be humanized, e.g., as described in EP 239 400; U.S. Pat. Nos. 6,602,503; 5,693,761; and 6,407,213, deimmunized, or otherwise modified to make it effectively human.

EP 239 400 (Winter et al.) describes altering antibodies by substitution (within a given variable region) of their complementarity determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody. Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; and 5,530,101; Tempest et al. (1991) Biotechnology 9:266-271 and U.S. Pat. No. 6,407,213.

Fully human monoclonal antibodies that bind to VEGF and VEGFR can be produced, e.g., using in vitro-primed human splenocytes, as described by Boemer et al. (1991) J. Immunol. 147:86-95. They may be prepared by repertoire cloning as described by Persson et al. (1991) Proc. Nat. Acad. Sci. USA 88:2432-2436 or by Huang and Stollar (1991) J. Immunol. Methods 141:227-236; also U.S. Pat. No. 5,798,230. Large nonimmunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol Today 2:371-378; and US 2003-0232333).

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-35), Hanseula, or Saccharomyces.

Antibodies, particularly full length antibodies, e.g., IgGs, can be produced in mammalian cells. Exemplary mammalian host cells for recombinant expression include Chinese Hamster Ovary (CHO cells) (including dihydrofolate reductase-negative CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NSO myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fc fusions) may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some proteins that include an Fc domain, the antibody/protein production system may be designed to synthesize antibodies or other proteins in which the Fc region is glycosylated. For example, the Fc domain of IgG molecules is glycosylated at asparagine 297 in the CH2 domain. The Fc domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the protein of interest, e.g., an antibody or Fc fusion protein. The protein can be purified from the milk, or for some applications, used directly.

Methods described in the context of antibodies can be adapted to other proteins, e.g., Fc fusions and soluble receptor fragments.

In certain implementations, nucleic acid antagonists are used to decrease expression of an endogenous gene encoding VEGF or a VEGFR. In one embodiment, the nucleic acid antagonist is an siRNA that targets mRNA encoding VEGF or a VEGFR. Other types of antagonistic nucleic acids can also be used, e.g., a dsRNA, a ribozyme, a triple-helix former, or an antisense nucleic acid. In some embodiments, nucleic acid antagonists can be directed to downstream effector targets of VEGFR activation.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA (e.g., an mRNA encoding VEGF or VEGFR) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding VEGF or VEGFR. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂)alkylaminocytosines and N⁴,N⁴—(C₁-C₁₂)dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂)alkylaminopurines and N⁶,N⁶—(C₁-C₁₂)dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂ -C₃₀ alkenyl, C₂ -C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. Nos. 4,987,071; 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y Acad. Sci. 660:27-36; and Maher (1992) Bioassays 14:807-15.

Artificial transcription factors can also be used to regulate expression of VEGF or a VEGFR. The artificial transcription factor can be designed or selected from a library, e.g., for ability to bind to a sequence in an endogenous gene encoding VEGF or VEGFR, e.g., in a regulatory region, e.g., the promoter. For example, the artificial transcription factor can be prepared by selection in vitro (e.g., using phage display, U.S. Pat. No. 6,534,261) or in vivo, or by design based on a recognition code (see, e.g., WO 00/42219 and U.S. Pat. No. 6,511,808). See, e.g., Rebar et al. (1996) Methods Enzymol 267:129; Greisman and Pabo (1997) Science 275:657; Isalan et al. (2001) Nat. Biotechnol 19:656; and Wu et al. (1995) Proc. Natl. Acad. Sci. USA 92:344 for, among other things, methods for creating libraries of varied zinc finger domains.

Optionally, an artificial transcription factor can be fused to a transcriptional regulatory domain, e.g., an activation domain to activate transcription or a repression domain to repress transcription. In particular, repression domains can be used to decrease expression of endogenous genes encoding VEGF or VEGFR. The artificial transcription factor can itself be encoded by a heterologous nucleic acid that is delivered to a cell or the protein itself can be delivered to a cell (see, e.g., U.S. Pat. No. 6,534,261). The heterologous nucleic acid that includes a sequence encoding the artificial transcription factor can be operably linked to an inducible promoter, e.g., to enable fine control of the level of the artificial transcription factor in the cell, e.g., an endothelial cell.

Administration

An agent described herein may be administered systemically or locally, e.g., topically. Topical administration of an agent described herein is the preferred route of administration. For topical application, the compositions of the present invention can include a medium compatible with a cell, explant or subject. Such topical pharmaceutical compositions can exist in many forms, e.g., in the form of a solution, cream, ointment, gel, lotion, shampoo, soap or aerosol. A wide variety of carrier materials can be employed in the composition of this invention such as alcohols, aloe vera gel, allantoin, glycerin, vitamin A and E oils, mineral oils, and polyethylene glycols. Other additives, e.g., preservatives, fragrance, sunscreen, or other cosmetic ingredients, can be present in the composition.

A preferred vehicle for topical delivery is liposomes. Liposomes can be used to carry and deliver an agent, e.g., a agent described herein, into a cell. Detailed guidance can be found in, e.g., Yarosh et al. (2001) Lancet 357: 926 and Bouwstra et al. (2002) Adv. Drug Deliv. Rev. 54 Suppl 1:S41

For systemic administration the agent may be administered via the orally route or the parenteral route, including subcutaneously, intraperitoneally, intramuscularly, intravenously or other route. For local administration, they are administered topically, transdermally, transmucosally, intranasally or other route. A cell may be contacted extracellularly or intracellularly with the agent, e.g., by microinjection or transfection. The agent may be applied and removed immediately, applied and not removed, and/or repeatedly applied with constant, increasing or decreasing frequency and/or at increasing or decreasing doses or concentrations. More than one route of administration may be used simultaneously, e.g., topical administration in association with oral administration. Examples of parenteral dosage forms include aqueous solutions of the active agent, in a isotonic saline, 5% glucose or other well-known pharmaceutically acceptable excipient. Solubilizing agents such as cyclodextrins, or other solubilizing agents well known to those familiar with the art, can be utilized as pharmaceutical excipients for delivery of the pigment modulating composition.

The composition may be provided as, e.g., a cosmetics, a medication or a skin care product. The composition can also be formulated into dosage forms for other routes of administration utilizing conventional methods. A pharmaceutical composition can be formulated, for example, in dosage forms for oral administration as a powder or granule, or in a capsule, a tablet (each including timed release and sustained release formulations), or a gel seal, with optional pharmaceutical carriers suitable for preparing solid compositions, such as vehicles (e.g., starch, glucose, fruit sugar, sucrose, gelatin and the like), lubricants (e.g., magnesium stearate), disintegrators (e.g., starch and crystalline cellulose), and binders (e.g., lactose, mannitol, starch and gum arabic). When the composition is an injection, for example, solvents (e.g., distilled water for injection), stabilizers (e.g., sodium edetate), isotonizing agents (e.g., sodium chloride, glycerin and mannitol), pH-adjusting agents (e.g., hydrochloric acid, citric acid and sodium hydroxide), suspending agents (e.g., methyl cellulose) and the like may be used.

The agent may contain other pharmaceutical ingredients, e.g., a second treatment for skin, e.g., a moisturizer, a sunscreen.

Kits

An agent described herein (e.g., VEGF antibody or an agent that modulates VEGF or VEGFR) can be provided in a kit. The kit includes (a) an agent, e.g., a composition that includes an agent, and (b) informational material. The informational material can be descriptive, instructional, marketing or other material that relates to the methods described herein and/or the use of VEGF or VEGFR for the methods described herein. For example, the informational material relates to acute UVB skin damage, e.g., sunburn.

In one embodiment, the informational material can include instructions to administer an agent described herein in a suitable manner to perform the methods described herein, e.g., in a suitable dose, dosage form, or mode of administration (e.g., a dose, dosage form, or mode of administration described herein). Preferred doses, dosage forms, or modes of administration are topical and percutaneous. In another embodiment, the informational material can include instructions to administer an agent described herein to a suitable subject, e.g., a human, e.g., a human having, or at risk for, acute UVB damage.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about VEGF or VEGFR and/or its use in the methods described herein. Of course, the informational material can also be provided in any combination of formats.

In addition to an agent described herein, the composition of the kit can include other ingredients, such as a solvent or buffer, a stabilizer, a preservative, a fragrance or other cosmetic ingredient, and/or a second agent for treating a condition or disorder described herein. Alternatively, the other ingredients can be included in the kit, but in different compositions or containers than an agent described herein. In such embodiments, the kit can include instructions for admixing an agent described herein and the other ingredients, or for using an agent described herein together with the other ingredients.

An agent described herein can be provided in any form, e.g., liquid, dried or lyophilized form. It is preferred that an agent described herein be substantially pure and/or sterile. When an agent described herein is provided in a liquid solution, the liquid solution preferably is an aqueous solution, with a sterile aqueous solution being preferred. When an agent described herein is provided as a dried form, reconstitution generally is by the addition of a suitable solvent. The solvent, e.g., sterile water or buffer, can optionally be provided in the kit.

The kit can include one or more containers for the composition containing an agent described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an agent described herein. For example, the kit includes a plurality of syringes, ampules, foil packets, or blister packs, each containing a single unit dose of an agent described herein. The containers of the kits can be air tight and/or waterproof.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a swab.

### EXAMPLES

### Example 1

### Enhanced Cutaneous Photosensitivity in VEGF Transgenic Mice

The minimal erythema dose (MED), a parameter for cutaneous photosensitivity, was determined 48 h after a single UVB irradiation of the dorsal skin with graded doses of UVB. Wild-type mice developed visible erythema after a dose of 7.2×10⁻² J/cm², whereas VEGF overexpressing transgenic mice showed dorsal skin erythema after a threshold dose of 3.6×10⁻² J/cm² with marked edema formation at doses of 7.2×10⁻² J/cm² and higher. Erythema formation and edema after a dose of 3.6×10⁻² j/cm² were also found in the ear skin of VEGF transgenic mice but not in wild type littermates. At 48 h after UVB irradiation with one MED, the ear skin of VEGF transgenic mice showed characteristic features of acute photodamage, including epidermal hyperplasia, single keratinocyte necrosis (“sunburn cells”), marked dermal edema, vessel dilation and inflammatory cell infiltration. In contrast, wild-type skin exposed to the identical UVB dose did not show any major histological changes.

Double-immunofluorescence stains for the endothelial cell membrane molecule CD31 and for BrdU revealed greatly enlarged cutaneous vessels at 48 h after UVB irradiation of VEGF transgenic mice, with pronounced proliferation of epidermal keratinocytes and of vascular endothelial cells. In contrast, CD31-positive vessels remained non-dilated in the skin of UVB-irradiated wild-type littermates, and no proliferating endothelial cells were detected.

Fourty-eight hours after UVB irradiation with 3.6×10⁻² J/cm², VEGF transgenic mice showed a significantly increased ear thickness, whereas no ear swelling was detected after the dose of 1.8×10⁻² J/cm² (P<0.01). Ear thickness returned to baseline levels at 7 days after irradiation. No ear swelling response was seen in wild-type mice after UVB irradiation with 3.6×10⁻² J/cm². Together, these findings revealed an increased photosensitivity in the skin of VEGF transgenic mice.

To investigate whether the enhanced erythema and vascular response observed in VEGF transgenic mice might have been mediated by enhanced cutaneous levels of VEGF, we next measured VEGF protein levels in skin lysates. Using a specific ELISA assay for murine VEGF, we found a significant up-regulation of VEGF protein levels in the UVB irradiated skin of VEGF transgenic mice at 48 hours after irradiation with 3.6×10⁻² J/cm² (138.1+13.0 pg/mg protein), as compared with non-irradiated transgenic skin (53.2+6.6 pg/mg) (P<0.05). In wild-type mice, UVB irradiation with an identical dose did not significantly increase cutaneous VEGF protein levels (25.8+7.3 pg/mg), as compared with non-irradiated skin (14.8+5.9 pg/mg). VEGF protein levels were significantly higher in VEGF transgenic mice than in wild-type mice (P<0.05).

### Example 2

### Systemic Blockade of VEGF Activity Reduces the Skin Sensitivity to Acute UVB Irradiation

To investigate whether VEGF is necessary for the acute cutaneous UVB response, we next treated wild-type mice by injection with a neutralizing anti-VEGF antibody 24 h before and 24 h after a single UVB irradiation. Marked tissue edema was detected in control IgG-treated mice 48 h after irradiation with 5.4×10⁻² J/cm² of UVB, but not in anti-VEGF antibody-treated mice. After a UVB dose of 7.2×10⁻² J/cm² or higher, tissue edema and inflammatory cell infiltration were slightly reduced in VEGF-antibody treated mice, and the UVB-induced damage of collagen fibers was suppressed, as compared with IgG treated mice. Immunofluorescence stains for CD31 revealed marked enlargement of dermal blood vessels at 48 h after UVB irradiation of IgG-treated control mice with 5.4×10⁻² J/cm², whereas no such changes were found in anti-VEGF-treated mice. These findings were confirmed by computer-assisted morphometric analysis of CD31-stained sections, revealing a significant suppression of vessel size (211.1±16.8 mm²; P<0.01) and tissue area covered by vessels (2.28±0.19%; P<0.05) in the anti-VEGF antibody treated mice, as compared with control IgG treated mice (vessel size 452.3±40.1 mm²; vessel area 3.14±0.29%). No differences in vessel density were observed between the treatment groups. Accordingly, the MED at 48 h after UVB irradiation was higher in anti-VEGF treated mice (7.8+2.08×10⁻² J/cm²) than in control IgG treated mice (6.0+1.04×10⁻² J/cm²). Similarly, systemic treatment with the anti-VEGFR-2 blocking antibody DC101 also reduced acute photosensitivity with an increase of the MED.

### Example 3

### Overexpression of VEGF Promotes Chronic UVB-Induced Skin Damage

We next investigated whether chronically elevated levels of cutaneous VEGF might also promote skin sensitivity to the damage induced by chronic UVB irradiation. After 10 weeks of a 3× weekly irradiation with a dose of 1.8×10⁻² J/cm², the skin of wildtype mice did not show any signs of chronic UVB damage and was comparable to non-irradiated skin. In contrast, pronounced formation of skin wrinkles was detected in all VEGF transgenic mice. Histological analysis revealed epidermal hyperplasia, inflammatory cell infiltration, tissue edema and degradation of the collagen matrix in chronically UVB-irradiated VEGF transgenic mice, but not in non-irradiated transgenic mice or in wild-type mice with or without irradiation. Moreover, degradation of elastic fibers was regularly detected in the chronically UVB-irradiated skin of VEGF transgenic mice, but not in non-irradiated transgenic mice or in wild-type mice with or without irradiation.

Macroscopically, the skin of chronically UVB-irradiated VEGF transgenic mice showed enhanced vascularization, as compared with non-irradiated transgenic skin or with wild-type skin. Double immuno-fluorescence stains for CD31 and VEGFR-2 demonstrated a highly increased number of enlarged CD31-positive/VEGFR-2-positive vessels in the upper dermis of chronically UVB-irradiated VEGF transgenic mice, whereas little or no VEGFR-2 expression was found on CD31-positive vessels in non-irradiated transgenic mice or in wild-type mice. Wildtype mice showed no increase in the number of CD31-positive vessels after chrnic UVB irradiation, whereas increased numbers of tortuous vessels were found in non-irradiated transgenic skin. Morphometric analysis of CD31-stained skin sections confirmed these findings, with a significant increase of the average vessel density (251.3+26.6 per mm²; P<0.01), the cutaneous area covered by vessels (8.36+0.85%; P<0.001), and the average vessel size (523.3+42.5 mm²; P<0.01) in chronically UVB-irradiated VEGF transgenic mice, as compared with UVB-irradiated wild-type mice (vessel density 104.8+7.6 per mm²; average vessel size 230.7+30.4 mm²; average vessel area 2.02+0.25%). Non-irradiated skin of VEGF transgenic mice showed enhanced vessel density, average vessel size, and relative skin area covered by vessels, as compared with non-irradiated wild-type skin.

We implanted VEGF165 slow-releasing pellets subcutaneously into FVB wild-type mice, followed by irradiation with a single UVB dose of 5.4×10⁻² J/cm² after 5 days. VEGF165 is the human homolog of murine VEGF164. Two days after UVB irradiation, histologic analyses revealed marked tissue edema in mice implanted with VEGF165-releasing pellets but only minor edema in sham-irradiated mice bearing VEGF-releasing pellets and in UVB-irradiated mice bearing control pellets. No major changes were seen in sham-irradiated mice bearing control pellets. Immunofluorescence stains for CD31 revealed pronounced neovascularization and vessel enlargement in mice bearing VEGF-releasing pellets 48 hours after UVB irradiation, whereas less pronounced vascular enlargement was found in sham-irradiated mice with VEGF pellets and in UVB-irradiated mice bearing control pellets. No major vascular changes were observed in sham-irradiated mice implanted with control pellets.

### EXAMPLE

In healthy mouse skin, we found VEGF164 to be the predominantly expressed VEGF isoform, compared with low-level mRNA expression of VEGF120 and VEGF188. Forty-eight hours and 4 days after a single exposure of mouse skin to a dose of 200 mJ UVB, expression of VEGF164 was significantly (P<0.001) up-regulated (day 2, 1.09±0.26; day 4, 0.98±0.19; nonirradiated, 0.48±0.05). We also found enhanced expression of VEGF120 (day 2, 0.58±0.11, P<0.01; day 4, 0.39±0.05, P<0.001) and VEGF188 (day 2, 0.47±0.05, P<0.001; day 4, 0.61±0.12, P<0.01); however, VEGF164 was the most strongly expressed isoform at all time points studied. VEGF mRNA expression levels returned to background levels of nonirradiated skin at 8 days after irradiation. Together, these results reveal that VEGF164 is the predominant VEGF isoform induced by UVB irradiation of the skin.

UVB Irradiation Regime

Eight-week-old male FVB wild-type mice or transgenic mice that overexpress VEGF-A164 in the epidermis under control of the human keratin 14 promoter were exposed to graded doses of a single UVB irradiation, using a bank of four equally spaced fluorescent lamps (Southern New England Ultraviolet, Branford, Conn.). See Detmar et al. (1998) J. Invest Dermatol. 1111:1-6. The height of the lamps was adjusted to deliver 0.35 mW/cm² at the dorsal skin surface. The minimal erythema dose (MED) was determined by irradiation of square areas of back skin with 7 different doses of UVB, ranging from 1.8×10⁻² J/cm² to 1.26×10⁻¹ J/cm² (n=5 per group). An additional skin area was sham irradiated. Erythema formation was evaluated daily for up to 9 days by 2 independent observers, and ear thickness was measured daily as described.

In additional experiments, wild-type mice were treated with 50 μg goat antimouse VEGF-neutralizing antibody (R&D Systems, Minneapolis, Minn.), or with 50 μg control isotype immunoglobulin G (IgG) by intraperitoneal injection 24 hours before and 24 hours after a single irradiation with 7 graded doses of UVB, as described in the previous paragraph (n=5 per group). Moreover, 8-week-old male VEGF transgenic mice and their wild-type littermates were irradiated 3 times weekly for 10 weeks with single doses ranging from 1.8 to 2.4×10⁻² J/cm² (n=10 per genotype). No acute sunburn reactions were observed. Control mice were sham irradiated. Samples of back skin were snap-frozen in ethanol dry ice or fixed in 4% paraformaldehyde in phosphate-buffered saline (PBS). The Massachusetts General Hospital Subcommittee on Research Animal Care approved all animal studies.

RT-PCR Analysis of VEGF Isoform Expression

Female albino hairless mice (n=30; Hoshino Laboratory Animals, Yashio, Japan) were exposed to single doses of UVB irradiation (200 mJ/cm²) at 8 weeks of age from Toshiba FL-20 SE fluorescent lamps (Toshiba, Tokyo, Japan) that deliver energy in the UVB range (280-340 nm), as described. 10 Mice were humanely killed on days 2, 4, 6, 8, and 13 (n=5 per time point) after irradiation. Total RNA was isolated from the dorsal skin of these mice and of 5 nonirradiated control mice using TRI reagent (Sigma, St Louis, Mo.). Thereafter, cDNA was reverse transcribed using the SuperScript first-strand synthesis system (Invitrogen, Carlsbad, Calif.). Vascular endothelial growth factor (VEGF) and G3PDH were amplified using Platinum Taq DNA polymerase (Invitrogen) for 30 cycles at 58° C. Polymerase chain reaction (PCR) products were fractionated by gel electrophoresis.

Enzyme-Linked Immunosorbent Assay for VEGF-A

Skin lysates were obtained from ear skin 48 hours after UVB irradiation or sham irradiation (n=3 per group). Tissues were homogenized, and murine VEGF-A levels were quantified by an enzyme-linked immunosorbent assay (ELISA) (Quantikine M; R&D Systems), as described. This ELISA also detected the transgenic product, murine VEGF164. Experiments were performed at least twice with comparable results. Statistical analysis was performed using the unpaired Student t test.

Immunofluorescence and Computer-Assisted Morphometric Vessel Analysis

Immunofluorescence analysis was performed on 5-μm frozen sections, as described previously,22 using a monoclonal rat antimouse CD31 antibody (BD Biosciences PharMingen, San Diego, Calif.), a monoclonal rat antimouse VEGF receptor (VEGFR)-2 antibody (BD Biosciences PharMingen), a rabbit anti-Ki-67 antibody (Novocastra Laboratories, Newcastle, United Kingdom), and corresponding secondary antibodies labeled with Alexa Fluor 488 or 594 (Molecular Probes, Eugene, Oreg.). Representative sections were obtained from the skin of UVB-irradiated and of sham-irradiated mice (n=5 per group) and were analyzed using a Nikon E-600 microscope (Nikon, Melville, N.Y.) equipped with a Plan Fluor 10× objective with an aperture of 0.30 (Nikon), a Plan Fluor 20× objective with an aperture of 0.50, and a Plan Fluor 40× objective of 0.75. The image medium was air, and images were acquired using Adobe Photoshop (Adobe Systems, San Jose, Calif.). Images were captured with a Spot 1.3.0 digital camera (Diagnostic Instruments, Sterling Heights, Mich.), and morphometric analyses were performed using IP-LAB software (Scanalytics, Fairfax, Va.). Three different fields of each section were examined at 10× magnification, and the number of vessels per square millimeter, the average vessel size, and the relative area occupied by blood vessels were determined in the dermis, in an area within 200 μm distance from the epidermal-dermal junction. The unpaired Student t test was used to analyze differences in microvessel density and vascular size. In addition, paraffin sections were obtained from the skin of the same mice, with routine hematoxylin-eosin staining, Trichrome staining, and Luna aldehyde fuchsin (LUNA) staining.

Subcutaneous Micropocket Assay

Eight-week-old male FVB mice were implanted with pellets containing or not containing recombinant human VEGF 165 (500 ng/pellet) (R&D Systems) subcutaneously by surgical incision. Pellets were prepared as previously described.27,28 Five days after implantation, mice were irradiated with a single UVB dose of 5.4×10⁻² J/cm². Control mice were sham irradiated (n=5 per each group). After 48 hours, back skin samples were snap-frozen or fixed in 4% paraformaldehyde for histologic analysis.

It was also found that, in humans, a single dose of UVB radiation results in the infiltration of elastase-producing leucocytes, in elastic fibre damage and in pronounced dermal angiogenesis with upregulation of VEGF expression and concomitant downregulation of TSP-1 expression in the hyperplastic epidermis. UVB irradiation of human skin induces an angiogenic switch that involves upregulation of VEGF and potent suppression of TSP-1 expression, indicating an important role of angiogenesis in the mediation of UVB-induced skin damage.

All publications, patent applications and patents cited herein are hereby incorporated by reference, including 60/559,300; US 2003-0008821; Hirakawa et al. (2005) Blood, 105:2392-2399, and Yano et al. (2005) B. J. Derm. 152:115-121. A number of embodiments of the invention have been described. Nevertheless, it will be understood that various modifications may be made without departing from the spirit and scope of the invention. Accordingly, other embodiments are within the scope of the following claims.

1. A method of identifying an agent that reduces acute UVB-induced skin damage, the method comprising: evaluating a test agent for the ability to reduce VEGF signaling, to thereby identify an agent that reduces acute UVB-induced skin damage.

2. The method of claim 1, wherein the test agent is evaluated for the ability to inhibit VEGF interaction with a cognate receptor.

3. The method of claim 1, wherein the test agent is evaluated for the ability to inhibit VEGFR-2 kinase activity.

4. The method of claim 1 wherein the test agent is a polypeptide, an antibody, a botanical extract, a carbohydrate, a lipid, a nucleic acid or a small molecule

5. A method of reducing acute UVB-induced skin damage in a subject, the method comprising:
administering to a subject having, or at risk for, acute UVB-induced skin damage, an agent that inhibits VEGF signaling.

6. The method of claim 5, wherein the agent is an anti-VEGF antibody.

7. The method of claim 6, wherein the agent is bevacizumab.

8. The method of claim 5, wherein the agent is pomegranate seed oil or grape seed extract.

9. The method of claim 5, wherein the agent is applied topically.

10. The method of claim 5, wherein the agent is formulated as a cosmetic composition.

11. The method of claim 5, wherein the agent is an anti-VEGF receptor antibody.

12. The method of claim 5, wherein the agent is an inhibitor of VEGF receptor tyrosine kinase activity.

13. The method of claim 12, wherein the agent is 4-[4-(1-Amino-1-methylethyl)phenyl]-2-[4-(2-morpholin-4-yl-ethyl)phenylamino]pyrimidine-5-carbonitrile; PTK-787/ZK222584; SU5416; SU11248; or [N-(4-bromo-2-fluorophenyl)-6-methoxy-7-[(1-methylpiperidin-4-yl)methoxy]quinazolin-4-amine].

14. The method of claim 5, wherein the subject has a sun burn.

15. The method of claim 5, wherein the subject is hyper-sensitive to sun burns.

16. The method of claim 5, further comprising increasing TSP-1 or TSP-2 activity.

17. A method comprising:
applying, to a skin region of a human subject, a protective amount of a VEGF signalling inhibitor; and exposing the subject to irradiation.

18. A method of monitoring a subject or cells from a subject, the method comprising: evaluating expression of one or more of VEGF, TSP-1 and interferon beta, wherein an increase in VEGF levels and a decrease in TSP-1 or interferon beta indicates that the subject or cells from the subject are at risk for or have been exposed to a skin damaging condition.
